(19) United States
(12) Patent Application Publication
(10) Pub. No.: US 2018/0214618 A1
(43) Pub. Date: Aug. 2, 2018

(54) Cupping syringe

(71) Applicant: Mariam Mostafa, Alexandria, VA (US)

(72) Inventor: Mariam Mostafa, Alexandria, VA (US)

(21) Appl. No.: 15/421,955

(22) Filed: Feb. 1, 2017

(51) Int. Cl. A61M 1/08; A61M 1/00

(52) U.S. Cl. CPC A61M 1/08; A61M 1/0009; A61H 2009/0064; A61H 2201/013; A61M 1/0068

(57) ABSTRACT

The disposable Cupping device is useful for wet and dry cupping therapeutic treatment. It consists of a Barrel like cup, plunger with at least one projections, and a Locking mechanism.

### TECHNICAL FIELD

The present invention relates to a wet and dry cupping set, where suction is created on particular points on the patient's body to withdraw blood or just creating suction by the use of a device that is shaped like a disposable syringe.

### BACKGROUND OF THE INVENTION

Cupping therapy is an alternative medicine that requires using suction in a cup. There are different cupping sets in the market such as Kangzhu which consists of 12 cups and a separate pistol to produce suction. There are many downfalls to these sets. It is a relatively large set to carry which is inconvenient pertaining to mobility. They are reusable, but the clients must clean them after each procedure to use them again. It is a troublesome process and some clients do a poor job of cleaning them while others refuse to clean them and prefer to buy a new set every session.

Some practitioners prefer buying hundreds of disposable cups and a few pistols. They use the same pistol on many different clients which may increase the risk of contamination.

Another set that is used is a Twist Vacuum Suction cupping set. These cups will create suction by twisting the handle, therefore, it does not have a pistol. Again, it is big to carry, hard to clean, and the twisting technique produces pain on the practitioner's hand.

In addition, there is a Rubber bulb cupping set which is inefficient because its suction power is weak. It is expensive and hard to sanitize.

Vaclok syringe is a syringe that carry fluid to or from a vein and it has locking mechanism. It is available in 10 cc, 20 cc, 30 cc and 60 cc. It is very expensive, and does not fit the purpose of cupping because it has a very small opening that attached to a needle.

### SUMMARY OF THE INVENTION

The device of the present invention is a combination of cupping portion of cupping device integrating a syringe like device to create a vacuum. It is disposable and inexpensive. It is a cup integrated with inexpensive disposable vacuum device which it can be shaped like syringe. The invention consists of a Barrel like a cup, a plunger with at least one locking projection, and a Lock. It provides a strong suction, easy to mold or formed, easy to use and sanitized.

### BRIEF DESCRIPTION OF THE DRAWING

Further features and advantages of the Cupping syringe disclosed herein will become apparent from the following and more particular description as illustrated in the accompanying drawings in which:

FIG. 1 is illustration of prior art, the Rubber bulb cup.

FIG. 2 is illustration of prior art, Kangzhu cups with pistol.

FIG. 3 is illustration of prior art, Twist vacuum suction cup.

FIG. 4 is illustration of prior art, Vaclok syringe.

FIG. 5 is the cup and the syringe separated.

FIG. 5a is a different possibility of a barrel attached to a flare cup.

FIG. 5b is another possibility of a barrel attached to a cone cup.

FIG. 6 is a plan view of the Cupping syringe in the initial state or unlock state.

FIG. 7 is a plan view of the Cupping Syringe when the plunger rod was pulled out to create suction and locked on the lightest level of pressure.

FIG. 8 is a plan view of the Cupping Syringe when it is attached to the body.

### DETAILED DESCRIPTION OF THE INVENTION

Referring now to the drawing in detail:

FIG. 1 is the rubber bulb cup, the vacuum is created by squeezing the rubber bulb R to pump the air out then the cup C gets attached on the body surface.

FIG. 2 is Kangzhu cup K and a separated pistol P, the pistol creates suction on the cup to get attached on the body surface.

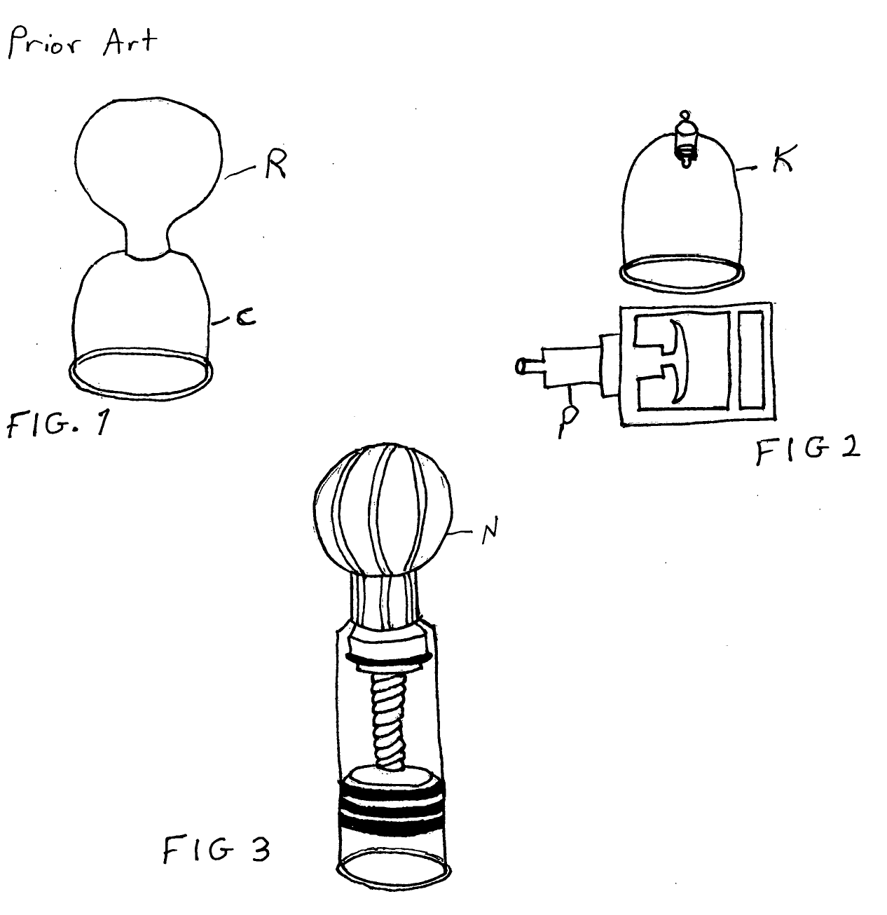

FIG. 3 is a twist vacuum suction cup, the suction is created by placing the cup on the skin and twisting the knob.

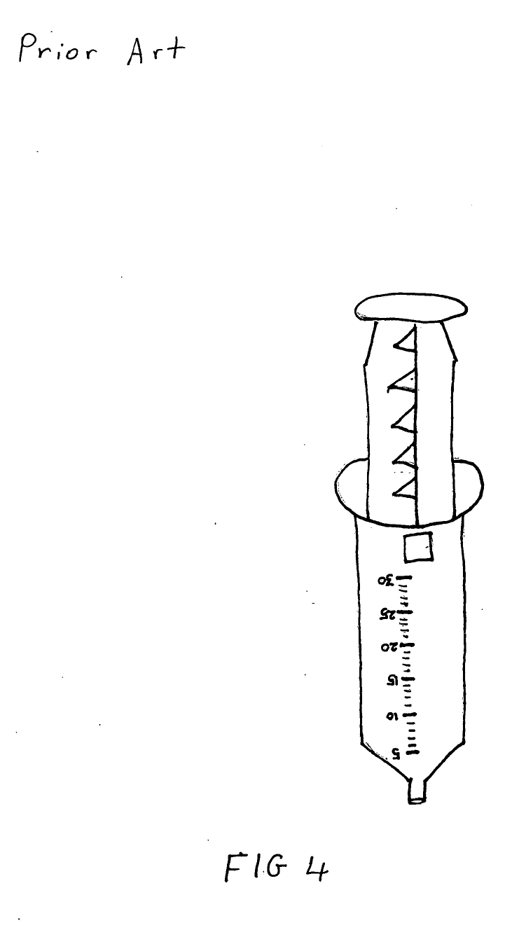

FIG. 4 is a Vaclok syringe, the barrel of the vaclok syringe is narrow and long. It is available in 10 cc, 20 cc, 30 cc and 60 cc. it is calibrated in tenth to make precise measurements of the quantity of the fluid that is to be given or removed. It has a small ending like a nozzle to get attached to a needle to be inserted into a vein or skin.

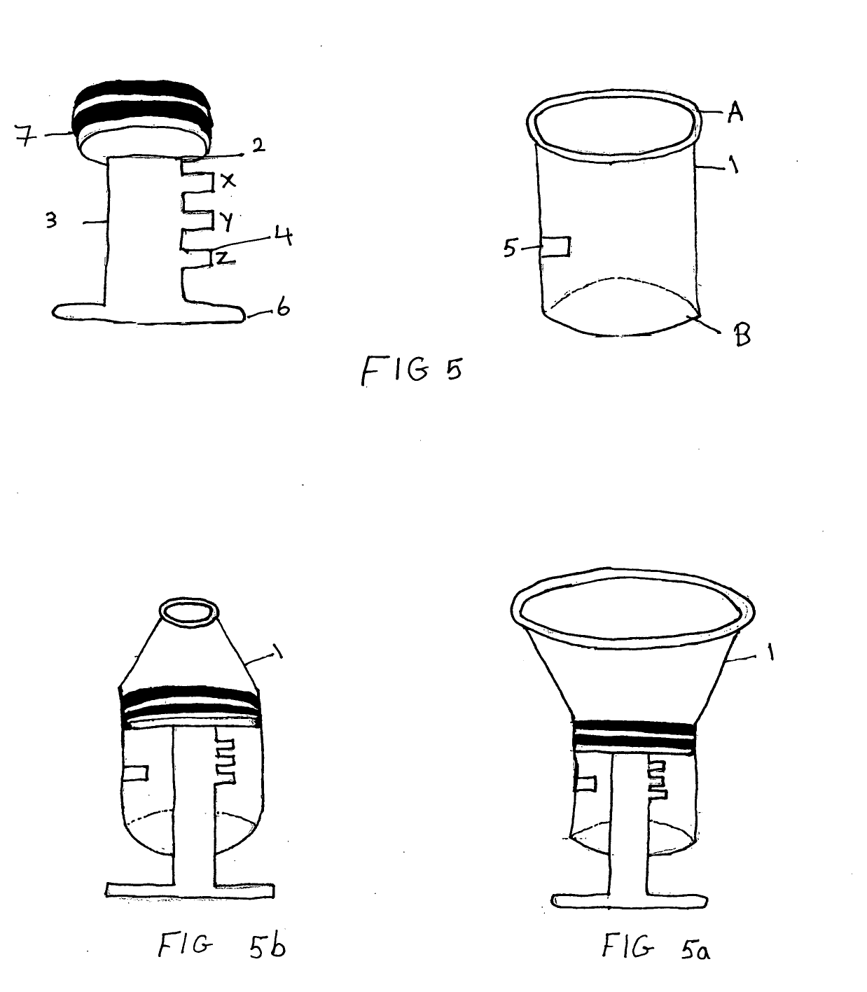

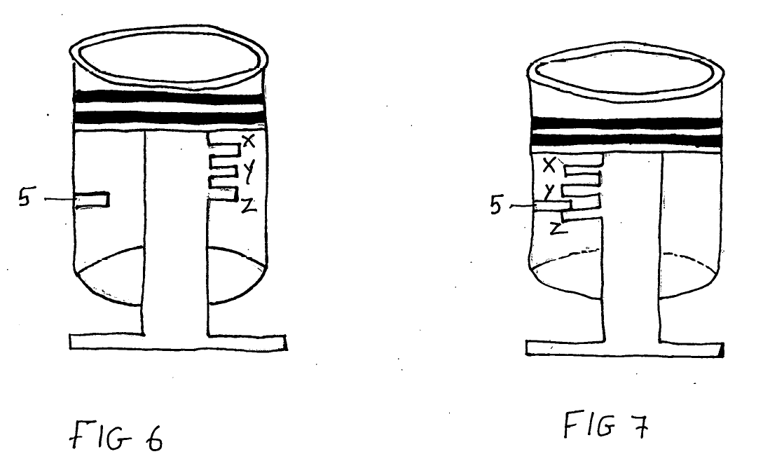

FIG. 5. Shows the Cupping syringe separated. The barrel 1 is short and wide, and has two opening with the same diameter on both ends. End A is round or oval shaped, smooth and wide to get attached to the body, while End B gets sealed by the rubber or any inexpensive sealer 7 that is attached on the top of the plunger tip 2. The barrel 1 has a locking mechanism 5 that it could be a pin or a hole. The plunger rod 3 sized to fit within the barrel 1 having a handle 6 and a tip 2. The seal is a rubber or inexpensive sealer 7 on the tip of the plunger rod 3 to engage the barrel 1 and provide a good seal for the vacuum. The plunger rod 3 has 3 protrusions, X, Y and Z, which engage a corresponding locking mechanism 5 on the barrel 1 to maintain a desirable position of the plunger when it is retracted. Protrusion X is the strongest suction, protrusion Z is the weakest suction, and protrusion Y is the medium suction.

FIG. 5a is the barrel 1 can be stretched to a cup with a flare opening.

FIG. 5b is the barrel 1 can be attached to a cone shaped cup.

FIG. 6 is the Cupping syringe in initial state which is not the locking position. The plunger rod 3 is inside the Barrel 1. The Barrel cup 1 and the plunger rod 3 could be made of clear, recycled plastic. The depth of the Barrel 1 could be 2.5″ but the diameter of the Barrel 1 could be as following: 2.5″, 2.25″, 2″, 1.75″, 1.5″, 1.25″, 1″, 0.75, 0.5″and 0.25″. These different in sizes allow the Barrel 1 to attach to different parts of the body.

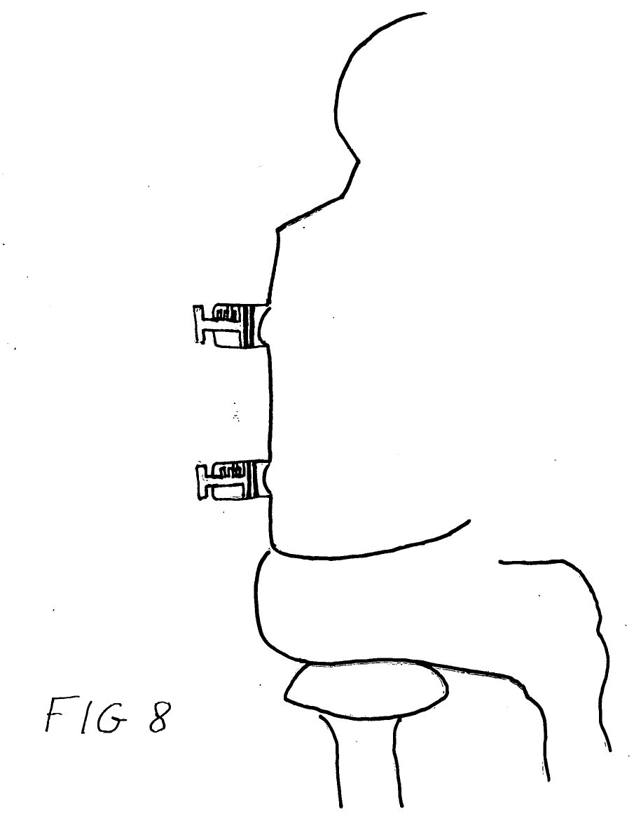

FIG.7 is showing the plunger rod 3 either pulled and locked or pulled, rotate and locked to the weakest level of suction Z.

FIG. 8 is showing the way the Cupping syringe attached to the body by placing the End A on the body, retracting the plunger rod 3 to create a vacuum, then plunger protrusions can engage the locking mechanism. The cup can be left on the body for at least 2 minutes in case of performing a Dry cupping treatment. The further step could be creating incisions on the skin at the cupping locations before placing the Cupping syringe on the body to withdraw the toxic blood in case of performing a Wet cupping treatment.

What I claim is:

1. A Cupping Syringe comprising:
A cylinder barrel having an opening on both ends with the same diameter;
A plunger sized to fit within the barrel having a handle and a tip;
A seal on the tip of the plunger to engage the barrel;
Where in the plunger has at least one protrusion to engage a corresponding locking mechanism on the barrel to maintain the position of the plunger when it is retracted.

1. The Cupping Syringe in claim 1 where in the two opening of the barrel are the same or different in sizes, the end that attached to the body could be bigger or smaller than the end that attached to the plunger.

2. The Cupping Syringe in claim 1 where in the opening that attached to the body could be round, oval or extended to a flare cup or a cone cup.

3. The opening in claim 3 where in it attached to the body must be smooth.

4. The Cupping Syringe in claim 1 where in the opening of the barrel is at least 0.25″ and the size of the barrel is at least 2.5″.

5. The Cupping Syringe in claim 1 where in the plunger has at least 1 protrusion.

6. The method of locking or unlocking the plunger in claim 6 could include rotating the plunger inside the barrel to engage the locking mechanism.

7. The Cupping Syringe in claim 1 where in the barrel has at least one pin or a hole as a locking mechanism.

8. The Cupping Syringe in claim 1, the barrel, the plunger, and the seal on the tip of the plunger are prepared of safe material to be in contact with human body;
The barrel and the plunger are made of durable transparent inexpensive recycled plastic;
The seal on the plunger is made of a rubber or any inexpensive sealer.

9. The Cupping Syringe in claim 1 where in the barrel is easily molded or formed;
The barrel and the plunger could be disposable or not disposable.

10. A method of using the Cupping Syringe as recited in claim 1 comprise:
Placing the opening of the syringe on a body;
Retracting the plunger to create a vacuum;
Lock the plunger in a desirable pressure;
Leave it for at least 2 minutes then the plunger gets unlocked and removed.

11. The method of using the Cupping Syringe in claim 11 further comprise;
The steps of creating shallow incisions on the surface of the skin at the cupping locations before placing the Cupping Syringe on the body to withdraw the toxic blood then the plunger gets unlocked and removed to clean the blood;
Repeat the process until no blood comes out.
